(19) United States
(12) Patent Application Publication
(10) Pub. No.: US 2026/0076889 A1
(43) Pub. Date: Mar. 19, 2026

(54) POLYESTER FILM-FORMING POLYMERS FOR ALCOHOLBASED SUNSCREEN FORMULATIONS

(71) Applicant: NOURYON CHEMICALS INTERNATIONAL B.V., Amsterdam (NL)

(72) Inventors: Hanamanthsa Shankarsa BEVINAKATTI, Somerset, NJ (US); Crystal Sonya PRIESTER, Rahway, NJ (US); Karen Lee WHITE, Bridgewater, NJ (US); Melissa Joy VITALE, Bedminster, NJ (US); Kristin Nicole GOLAS, Flemington, NJ (US)

(73) Assignee: NOURYON CHEMICALS INTERNATIONAL B.V., Amsterdam (NL)

(21) Appl. No.: 19/106,462

(22) PCT Filed: Aug. 25, 2023
(86) PCT No.: PCT/EP2023/073437

§ 371 Date: Feb. 25, 2025

Related U.S. Application Data
(60) Provisional application No. 63/401,220, filed on Aug. 26, 2022.

(51) Int. Cl. A61K 8/86; A61K 8/04; A61K 8/31; A61K 8/34; A61K 8/35; A61K 8/37; A61K 8/40; A61Q 17/04

(52) U.S. Cl. CPC A61K 8/86; A61K 8/046; A61K 8/31; A61K 8/34; A61K 8/35; A61K 8/37; A61K 8/40; A61Q 17/04; A61K 2800/95

(57) ABSTRACT

A sunscreen formulation includes (a) a volatile solvent; and (b) at least one film-forming polymer that is the reaction product of (i) at least one polyglycerol; (ii) at least one diacid having 4-30 carbon atoms; and (iii) at least one fatty acid having 8-30 carbon atoms. The sunscreen formulation is used in a method of protecting skin against damaging effects of the sun.

### CROSS-REFERENCE TO RELATED APPLICATIONS

This application is a U.S. National-Stage entry under 35 U.S.C. § 371 based on International Application No. PCT/EP2023/073437, filed Aug. 25, 2023, which was published under PCT Article 21(2) and which claims the benefit of U.S. Provisional Application No. 63/401,220, filed Aug. 26, 2022, which are all hereby incorporated in their entirety by reference.

### FIELD OF THE DISCLOSURE

The present disclosure relates to film-forming polymers for sunscreen formulations and to methods of using the sunscreen formulations.

### BACKGROUND

Polymers used in sunscreen applications have conventionally been made using synthetic materials. In order for the polymers to be suitable in such sunscreen applications, they must be soluble in alcohol-based systems, and in the case of aerosol-based sunscreen sprays, they must also be compatible with the propellant. Conventional synthetic polymers are generally inexpensive and provide acceptable performance. However, because they are not made from renewable resources and are not biodegradable, conventional synthetic polymers are not sustainable. In addition, replicating the cost and performance of synthetic polymers is not easy.

Accordingly, there is a need for biodegradable polymers made from renewable sources that provide equal to or better performance at comparable costs than their synthetic alternatives and that are soluble in alcohol-based systems, such as ethanol-based systems and optionally, that are compatible with propellants, such as dimethyl ether.

Suncare formulations based on biodegradable polyesters are the subject of recent publications: US 2021/0259930; US 2021/0259945; and US 2021/0259946.

US 2021/0259946, in particular, describes the preparation of polyesters for use in water-resistant anhydrous suncare preparations by, for example, a process comprising the substantially non-sequential reaction of the following components: (i) at least one polyglycerol; (ii) at least one dimer acid; and (iii) at least one fatty acid having 8-30 carbon atoms, wherein (iii) and (i) are in a molar ratio of less than 2:1. Preference is given to the use of C36 dimer acid. Some of the polyesters prepared in this manner are not compatible in ethanol spray formulations or do not provide the desired SPF performance when delivered from ethanol systems.

In general, the compatibility of bio-based film-forming polymers with alcohol-based, especially ethanol-based, sunscreen formulations, remains a significant problem for the formulator and there remains a significant need for bio-based film-forming polymers that are readily biodegradable yet at the same time are soluble in and compatible with alcohol-based systems. In addition, other objects, desirable features and characteristics will become apparent from the subsequent summary and detailed description, and the appended claims and this background. It is to be appreciated that all numerical values as provided herein, save for the actual examples, are approximate values with endpoints or particular values intended to be read as “about” or “approximately” the value as recited.

### BRIEF SUMMARY

The present disclosure provides a sunscreen formulation comprising:
(a) a volatile solvent; and
(b) at least one film-forming polymer that is the reaction product of
   (i) at least one polyglycerol;
   (ii) at least one diacid having 4-30 carbon atoms; and
   (iii) at least one fatty acid having 8-30 carbon atoms.

The present disclosure also provides a method of protecting a user to be exposed or already exposed to sunlight from the damaging effects of exposure to sunlight, wherein the method includes the step of applying the sunscreen formulation to skin.

### DETAILED DESCRIPTION

The following detailed description is merely exemplary in nature and is not intended to limit the present disclosure or the application and uses of the present disclosure. Furthermore, there is no intention to be bound by any theory presented in the preceding background of the present disclosure or the following detailed description.

It is to be appreciated that all numerical values as provided herein, save for the actual examples, are approximate values with endpoints or particular values intended to be read as “about” or “approximately” the value as recited.

It is an object of the present disclosure to provide film-forming polymers that are readily biodegradable, exhibit excellent compatibility with alcohol-based formulations, especially ethanol-based formulations, and, therefore, can be formulated into stable sunscreen formulations that provide biodegradability.

In US 2021/0259946, a preference was stated for the use of C36 or higher dimer acid to provide greater hydrophobic content for waterproofing.

Surprisingly, it was discovered that the use of diacids as herein disclosed results in formulations that have excellent ethanol solubility and a high degree of biodegradability.

Thus, in one embodiment, the present disclosure relates to a sunscreen formulation comprising:
(a) a volatile solvent; and
(b) at least one film-forming polymer that is the reaction product of the following components: (i) at least one polyglycerol; (ii) at least one diacid having 4-30 carbon atoms; and (iii) at least one fatty acid having 8-30 carbon atoms; but wherein the at least one film-forming polymer is not formed from dimer acid.

In another embodiment, the present disclosure relates to a sunscreen formulation comprising:
(a) volatile solvent; and
(b) at least one film-forming polymer that is the reaction product of the following components: (i) at least one polyglycerol; (ii) at least one diacid having 4-30 carbon atoms; (iii) at least one fatty acid having 8-30 carbon atoms; and (iv) at least one dimer acid.

In a typical embodiment, the diacid has 4-24 carbon atoms. Suitable diacids include butanedioic acid (e.g., succinic acid), pentanedioic acid (e.g., glutaric acid), hexanedioic acid (e.g., adipic acid), heptanedioic acid (e.g., pimelic acid), octanedioic acid (e.g., suberic acid), nonanedioic acid (e.g., azelaic acid), decanedioic acid (e.g., sebacic acid), undecanedioic acid, dodecanedioic acid, tridecanedioic acid (e.g., brassylic acid), hexadecanedioic acid (e.g., thapsic acid), heneicosanedioic acid (e.g., japanic acid), docosanedioic acid (e.g., phellogenic acid), crocetin, butenedioic acid (e.g., maleic acid, fumaric acid), butynedioic acid (e.g., acetylenedicarboxylic acid), pentenedioic acid (e.g., glutaconic acid, glutinic acid, citraconic acid, mesaconic acid, itaconic acid), hexenedioic acid (e.g., muconic acid), decenedioic acid, dodecanedioic acid (e.g., traumatic acid), and mixtures thereof.

In another typical embodiment, the diacid corresponds to the formula (I):

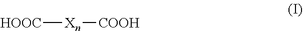

wherein
X represents —CH₂—; and
n represents 2-28, typically 8-22, more typically 10-22; and
wherein X₁ is optionally substituted by one or more —COOH groups, typically 1 or 2-COOH groups.

In another typical embodiment, the diacid corresponds to the formula (II):

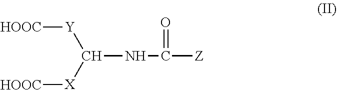

wherein
X and Y independently represent hydrocarbyl having 1-3 carbon atoms, typically, alkyl having 1-3 carbon atoms;
Z represents hydrocarbyl having 1-20 carbon atoms; and
X, Y, and Z are optionally substituted by additional carboxylic acid functionality.
   Suitable diacids falling under this embodiment include N-lauryl glutamic acid, N-stearoyl glutamic acid, and the like.

In a more typical embodiment, the diacid has 10-24 carbon atoms.

In an even more typical embodiment, the diacid has 12-24 carbon atoms.

In a most typical embodiment, the diacid is chosen from dodecanedioic acid, sebacic acid, N-stearoyl glutamic acid, and mixtures thereof.

In a typical embodiment, the film-forming polymer is prepared using dimer acid in addition to diacid. In this regard, it was discovered that the inclusion of dimer acid adds hydrophobicity to the polymer molecule and, thus, enhances the waterproofing.

In a more typical embodiment, the film-forming polymer is prepared using C36 or higher dimer acid, particularly C36 dimer acid, in addition to diacid.

By “dimer acid,” as the term is used in this disclosure, it means a dicarboxylic or higher carboxylic acid prepared by dimerizing unsaturated acids, particularly dimerized fatty acids prepared by dimerizing unsaturated fatty acids. This includes, of course, preparations of dimerized unsaturated acids that include not only dimers, but also trimers, tetramers, etc.

By “diacid,” as the term is used in this disclosure, it means a dicarboxylic or higher carboxylic acid not prepared by dimerizing unsaturated acid.

In an even more typical embodiment, the film-forming polymer is prepared using up to 15 wt % C36 or higher dimer acid, typically 1-10 wt %, particularly 5-6 wt % C36 dimer acid, in addition to diacid, the wt % in each case being based on a total weight of the components making up the film-forming polymer.

In an especially typical embodiment, the diacid is chosen from dodecanedioic acid, sebacic acid, N-stearoyl glutamic acid, and mixtures thereof with each other and/or with at least one C36 or higher dimer acid.

In another especially typical embodiment, the diacid is chosen from dodecanedioic acid, sebacic acid, N-stearoyl glutamic acid, and mixtures thereof, and does not comprise any C36 or higher dimer acid.

In another embodiment, the diacid is hydrogenated.

In yet another embodiment, the diacid is chosen from dodecanedioic acid, sebacic acid, N-stearoyl glutamic acid, and mixtures thereof with each other and/or with at least one C36 or higher dimer acid, wherein at least one of the diacids and/or the diacids is hydrogenated.

The polyglycerol can be any oligocondensation product of glycerol. In one embodiment, the polyglycerol has the formula (III):

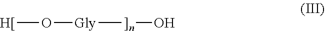

where each Gly is independently the residue of a molecule of glycerol after removal of two hydroxyl groups; and n is (an average of) from 2 to 10.

Generally, most of the groups Gly will be of the formula: —CH₂—CHOH—CH₂—, although residues comprising etherification at the secondary or even tertiary hydroxyl groups are considered to be within the scope of “Gly” and, thus, may also be present. Examples of oligoglycerols include diglycerol, triglycerol, tetraglycerol, pentaglycerol, hexaglycerol, heptaglycerol, octaglycerol, nonaglycerol, decaglycerol, and mixtures of these. Particularly useful polyglycerols are those of the formula (III) where n is particularly from 2 to 7, more particularly from 2 to 5 and especially 2, 3 or 4, or mixtures of oligoglycerols in these ranges.

Particularly suitable polyglycerols comprise a mixture of oligoglycerols having the following oligomer distribution:
Glycerol: 0 to 30% by weight, typically 0 to 20% by weight, most typically 0 to 15% by weight;
Diglycerol: 10 to 40% by weight, typically 15 to 35% by weight, most typically 20 to 32% by weight;
Triglycerol: 10 to 65% by weight, typically 15 to 60% by weight, most typically 18 to 55% by weight;
Tetraglycerol: 2 to 25% by weight, typically 5 to 20% by weight, most typically 8 to 20% by weight;
Pentaglycerol: 0 to 15% by weight, typically 0 to 10% by weight, most typically 0 to 5% by weight;
Hexaglycerol: 0 to 15% by weight, typically 0 to 10% by weight, most typically 0 to 5% by weight;
Heptaglycerol: 0 to 10% by weight, typically 0 to 5% by weight, most typically 0 to 3% by weight;
Octaglycerol: 0 to 10% by weight, typically 0 to 5% by weight, most typically 0 to 3% by weight;
Nonaglycerol: 0 to 5% by weight, typically 0 to 3% by weight, most typically 0 to 2% by weight;
Decaglycerol: 0 to 5% by weight, typically 0 to 3% by weight, most typically 0 to 2% by weight wherein all weight percentages are based on a total content of the polyglycerol.

In one embodiment, the polyglycerol comprises the following oligomer distribution:
Glycerol: 0 to 30% by weight;
Diglycerol: 15 to 40% by weight;
Triglycerol: 10 to 55% by weight;
Tetraglycerol: 2 to 25% by weight;
Pentaglycerol and higher components: 0 to 15% by weight;
wherein all weight percentages are based on a total content of the polyglycerol.

In one embodiment, the polyglycerol is composed of at least 40% by weight, or at least 45% by weight, or at least 50% by weight, based on a total weight of the polyglycerol, of a combination of diglycerol and triglycerol.

In one embodiment, the polyglycerol is composed of at least 20% by weight, or at least 25% by weight of diglycerol; at least 15% by weight, or at least 18% by weight of triglycerol; at least 10% by weight, or at least 12% by weight of tetraglycerol; wherein all weight percentages are based on a total content of the polyglycerol.

A particularly typical polyglycerol comprises at least 25% by weight diglycerol, at least 45% by weight triglycerol, and at least 10% by weight tetraglycerol.

Analysis of any such polyglycerol composition can be done to determine a median, mean, or “average” polyglycerol number. Oligoglycerol examples above with both narrow and broad distributions can be equally designated as polyglycerol-3, as this is the closest integer to the mean and/or median.

The fatty acid reactants are typically fatty monoacids and act as end-caps of the polymerization reaction, provide tunable hydrophobic content, and contribute to polymer properties. While all monoacids having 8-30 carbon atoms can be used, especially monoacids having 12-30 carbon atoms, C18 or greater monoacids are preferred to provide greater hydrophobic content for waterproofing. These can include naturally occurring or refined fatty acids, such as hydrolyzed rapeseed oil, sunflower oils etc. However these include both lower and higher MW chains.

Useful fatty monoacids can be linear, branched, saturated, unsaturated, and aromatic materials with acidity provided by carboxylic acid moieties. Useful acids include Caprylic acid (C8), Pelargonic acid (C9), Capric acid (C10), Undecylic acid (C11), Lauric acid (C12), Tridecylic acid (C13), Myristic acid (C14), Pentadecylic acid (C15), Palmitic acid (C16), Margaric acid (C17), Stearic acid (C18), Isostearic acid (C18), Nonadecylic acid (C19), Arachidic acid (C20), Behenic acid (C22), and Lignoceric acid (C24). Lower molecular weight organic acids could also be used instead of the fatty monoacid including Butyric acid (C4), Valeric acid (C5), Caproic acid (C6), Enanthic acid (C7). The raw materials can be derived from tall oil and rapeseed oil, but other natural sources including flaxseed, soybean, pumpkin, walnut can be used. For example, in one typical embodiment, the fatty acid utilized is rape fatty acid.

Comparing stearic and isostearic acid shows that branching leads to low melting point and results in a low viscosity at room temperature for isostearic acid, vs a solid material for stearic acid. This lower viscosity can be helpful in the materials handling of the raw materials and also in enabling esters made with this acid to retain liquid properties. Isostearic acid is available as a reaction byproduct in the creation of dimer acid described above.

Another route to obtaining a liquid product is to use unsaturated linear and branched fatty monoacids. These unsaturated acids can include Palmitoleic acid (C16:1), Vaccenic acid (C18:1), Oleic acid (C18:1), Elaidic acid (C18:1), Linoleic acid (C18:2), Linolelaidic acid (C18:2), α-Linolenic acid (C18:3), γ-Linolenic acid (C18:3), Stearidonic acid (C18:4), Paullinic acid (C20:1), Gondoic acid (C20:1), Dihomo-γ-linolenic acid (C20:3), Mead acid (C20:3), Arachidonic acid (C20:4), Eicosapentaenoic acid (C20:5), Erucic acid (C22:1), Docosatetraenoic acid (C22:4), Cervonic acid (C22:6), and Nervonic acid (C24:1). As is well-known to persons skilled in the art, the designation “CX:Y” means the length of the carbon chain is X carbon atoms; and there are Y number of double bonds in the chain.

All of these acids and mixtures thereof provide hydrophobicity when esterified with polyglycerol. Saturated fatty acids also will provide less manufacturing side reaction and greater long-term finished product storage due to oxidation of unsaturated bonds that can lead to color and other side products.

In one embodiment, the fatty acid is stearic acid, a straight-chain saturated C18 fatty acid, or oleic acid which is a monounsaturated C18. However, points of unsaturation can provide later oxidative instability and straight linear C18 fatty acid can lead to polymer crystallization.

In another embodiment, the fatty acid is chosen from rape fatty acid, stearic acid, behenic acid, isostearic acid, lauric acid, and mixtures thereof.

In an especially typical embodiment, the waterproofing polymer is a substantially or completely non-sequential reaction product of the following components: (i) at least one polyglycerol; (ii) at least one diacid having 4-30 carbon atoms; and (iii) at least one fatty acid having 8-30 carbon atoms.

By “substantially non-sequential reaction product” is meant the product is produced by substantially non-sequential reaction of the reacting components (i)-(iii) (and (iv) if dimer acid is utilized). By substantially non-sequential reaction of the reacting components (i)-(iii) (and (iv) if dimer acid is utilized) is meant substantially the total content of each of the reactants (i)-(iii) (and (iv) if dimer acid is utilized) to be reacted is added to the reaction vessel prior to commencing the reaction. This processing is distinct, for example, from that described in U.S. Pat. No. 6,242,499, wherein polyglycerol is esterified with fatty acid in a first step and, after most, or all, of the fatty acid has reacted, in a second step polyfunctional carboxylic acid is added and the esterification reaction is continued. In one embodiment of the present disclosure, the total content of each of the reactants (i)-(iii) (and (iv) if dimer acid is utilized) to be reacted is added to the reaction vessel prior to commencing the reaction, i.e., the reaction is completely non-sequential, and the polymer is a completely non-sequential reaction product of the components (i)-(iii) (and (iv) if dimer acid is utilized). In other embodiments, at least 60-100%, or 75-100%, or 80-100%, or 85-100%, or 90-100%, or 95-100%, or 97-100% of each of the reactants (i)-(iii) (and (iv) if dimer acid is utilized) is added to the reaction vessel prior to commencing the reaction.

In another especially typical embodiment, the waterproofing polymer is a substantially or completely non-sequential reaction product of the following components: (i) at least one polyglycerol; (ii) at least one diacid having 4-30 carbon atoms; (iii) at least one fatty acid having 8-30 carbon atoms; and (iv) at least one dimer acid, particularly dimer acid having at least 36 carbon atoms.

In another especially typical embodiment, the waterproofing polymer is a substantially or completely non-sequential reaction product of the following components: (i) at least one polyglycerol; (ii) at least one diacid having 4-30 carbon atoms; and (iii) at least one fatty acid having 8-30 carbon atoms; but does not include (iv) any dimer acid.

It has been found that the best waterproofing properties of the polymer result when the polyglycerol includes excess OH groups, relative to the acid groups added by the combination of diacid and fatty acid materials. As described above, a wide variety of polyglycerols have been used in blended form and deliver the unique performance given by this material.

In one embodiment, the components reacted are in a mole ratio of 1 mole polyglycerol, 0.5 to 1 mol dimer acid, and 0.2 to 1.7 mole fatty acid.

In another embodiment, the components reacted are in a mole ratio of 1 mole polyglycerol, 0.5 to 0.75 mol dimer acid, and 0.4 to 1.35 mole fatty acid.

In another embodiment, the components reacted are in a mole ratio of 1 mole polyglycerol, 0.5 to 0.7 mol dimer acid, and 0.65 to 1 mole fatty acid.

In one embodiment, the components are reacted in a mole ratio of 1 mole of C36 or higher dimer acid and 1-20 mole C30 or lower diacid.

In another embodiment, the components are reacted in a mole ratio of 1 mole of C36 or higher dimer acid and 1-15 mole C30 or lower diacid.

In another embodiment, the components are reacted in a mole ratio of 1 mole of C36 or higher dimer acid and 2-10 mole C30 or lower diacid.

By adjusting the fatty acid end-cap mole ratio and balancing the amount of polyglycerol, dimer acid and lower diacid, it is possible to control the degree of dimer acid-polyglycerol-extension and end-capping so that cross-linking, for example, via trimer acid, leads to much higher viscosities.

The disclosed sunscreen formulations additionally comprise a volatile solvent. Examples of volatile solvents include one or more of alcohols, such as methanol, ethanol and isopropanol; volatile hydrocarbons, such as isooctane, isododecane, and isohexadecane; volatile aldehydes; volatile silicones; and volatile ketones, such as acetone and methyl ethyl ketone. Hydrofluoro-olefins may also be used as a carrier solvent in the formulations.

In a typical embodiment, the volatile solvent is an alcohol-based solvent system, wherein the alcohol-based solvent system comprises at least one C₁₋₆ straight or branched chain alcohol.

In another typical embodiment, the volatile solvent is a ketone.

In an especially typical embodiment, the volatile solvent is chosen from ethanol, methanol, isopropanol, acetone, and mixtures thereof.

In another especially typical embodiment, the sunscreen formulations comprise ethanol.

In a more typical embodiment, the sunscreen formulations comprising ethanol are anhydrous. The terms “non-aqueous” and “anhydrous” are used interchangeably herein and refer to compositions including less than about 10% by weight water, especially less than about 5% by weight water, or less than 1% by weight water, or even 0% water.

In a most typical embodiment, the sunscreen formulation comprises ethanol but not any other alcohol or water.

In an alternative embodiment, where water is present, this small amount of water may be desirable, for example as a processing aid or co-solvent. In certain example embodiments, the water contents of the compositions will be no greater than about 9% water so as to prevent the active to phase-separate or precipitate out of solution. Those of ordinary skill in the art will recognize that different actives have different tolerances for water in solution and will adjust water content accordingly.

In an embodiment, the polymer is fully soluble in an ethanol or a predominately ethanol mixture with 0-20 wt %, typically 0-10% water, a cosolvent and/or UV sunscreen active agents.

In some cases, the polymer may be insoluble in ethanol after 24 hours.

However, in a more typical embodiment, the polymer is soluble in a formulation comprising one or more sunscreen active agents, one or more emollients, and ethanol immediately after the polymer is introduced to the formulation. The phrase “immediately after” as used throughout this disclosure means an hour or less, typically a half-hour or less, most typically 15 minutes or less.

In another more typical embodiment, the polymer remains fully soluble in said formulation after 24 hours after the polymer is incorporated into the formulation.

In an especially typical embodiment, the polymer is:
(a) fully soluble in said formulation immediately after the polymer is incorporated into the formulation; and
(b) fully soluble in said formulation after 24 hours after the polymer is incorporated into the formulation.

In one embodiment, the sunscreen formulations further comprise a propellant.

In certain embodiments of the subject disclosure, the disclosed formulations can be stored in containers under pressure by combination with the propellant and the disclosure extends to such containers under pressure including the disclosed formulations. The formulations thus stored can be applied by opening a valve in the container releasing the propellant and the composition, typically in a spray or mist. The propellant used in the composition may be any suitable gas, or combination of gasses, that can be compressed or liquefied within a dispensing spray canister, which expand or volatilize to vapor or gas form upon exposure to ambient temperature and pressure conditions to deliver the composition in an aerosol form. Suitable propellants include hydrocarbons having 1 to 5 carbon atoms, including but not limited to methane, ethane, propane, isopropane, butane, isobutane, butene, pentane, isopentane, neopentane, and pentene, hydrofluorocarbons (HFCs), chlorofluorocarbons (CFCs), hydrofluoro-olefins (HFOs), nitrogen, ethers including dimethyl ether, and any mixtures thereof. Those of ordinary skill in the art recognize that in a closed container such as an aluminum can or glass bottle, propellants such as dimethyl ether condense to the liquid state at ambient temperature. Thus, the composition in the aerosol container is a liquid formulation that can include dissolved propellant, undissolved liquid propellant and gaseous propellant. All of this is under pressure due to the vapor pressure of the propellant. In the practice of this aspect of the subject disclosure, the propellant can be present in an amount up to about 90 weight percent, typically from about 2 weight percent to about 50 weight percent, and more typically about 5 weight percent to about 40 weight percent, more typically at about 30 weight percent, based on the total weight of the aerosol composition.

In another embodiment, the propellant is a hydrocarbon-derived ether.

In another embodiment, the propellant is isobutane.

In a typical embodiment, the polymer is fully compatible with isobutane.

In an especially typical embodiment, the polymer is:
(a) fully soluble in a formulation comprising one or more sunscreen active agents, one or more emollients, and ethanol immediately after the polymer is incorporated into the formulation;
(b) fully soluble in said formulation after 24 hours after the polymer is incorporated into the formulation; and
(c) fully compatible with isobutane.

In one embodiment, the personal care formulation disclosed here is dispensed from a bag-on-valve device. Generally, bag-on-valve devices comprise a spray can fitted with an aerosol valve and comprising a welded bag. The product is placed inside the bag while the propellant is filled in the space between the bag and the can. The product is dispensed by the propellant simply squeezing the bag when the spray button is pressed.

Generally, the compositions according to the disclosure are prepared as non-aqueous, volatile solvent-based compositions. However, in some embodiments, the compositions comprise a single liquid phase that may further comprise dispersed particulates, for example, UV active agents in particulate form.

In one embodiment, the film-forming polymer is incorporated into the sunscreen formulations to impart waterproofing properties thereto.

In one embodiment, the waterproofing polymer is incorporated into sunscreen formulations in an amount of 0.1 to 10 wt % based on a total weight of the formulation.

In another embodiment, the waterproofing polymer is incorporated into sunscreen formulations in an amount of 0.5 to 5 wt % based on a total weight of the formulation.

In yet another embodiment, the waterproofing polymer is incorporated into sunscreen formulations in an amount of 1 to 3 wt % based on a total weight of the formulation.

The disclosed sunscreen formulations additionally comprise at least one sunscreen active agent. For purposes of the present disclosure, a “sunscreen active agent” is a material, used singly or in combination with other such materials, that is regarded as acceptable for use as an active sunscreening ingredient based on its ability to absorb UV radiation. Such compounds are generally described by their ability to act as UV active agents and their performance in different spectra regions describes as UV-A, UV-B, or UV-A/UV-B. Approval by a regulatory agency is generally required for inclusion of active agents in formulations intended for human use. Those active agents which have been or are currently approved for sunscreen use in the United States include organic and inorganic substances including, without limitation, para aminobenzoic acid, avobenzone, cinoxate, dioxybenzone, homosalate, menthyl anthranilate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum. Examples of additional sunscreen actives that have not yet been approved in the U.S. but are allowed in formulations sold outside of the U.S. include ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. However, as the list of approved sunscreens is currently expanding, those of ordinary skill will recognize that the disclosure is not limited to sunscreen active agents currently approved for human use but is readily applicable to those that may be allowed in the future.

In various embodiments, sunscreen active agents include, again without limitation, benzophenones, for example, Benzophenone-3 (BP3) and Benzophenone-4 (BP4); Salicylates, for example, Homosalate (HMS) and 2-ethylhexyl salicylate (EHS); p-Aminobenzoic acid and derivatives, for example, Ethylhexyl dimethyl PABA (OD-PABA) and 4-p-aminobenzoic acid (PABA); Benzimidazole derivatives, for example, Phenylbenzimidazole sulfonic acid (PMDSA) and Disodium phenyl dibenzimidazole tetrasulfonate (bisdisulizole disodium); Triazines, for example, Ethylhexyltriazone (OT), Diethylhexyl butamido triazone (DBT), and Bis-ethylhexyloxyphenol methoxyphenyl triazine (EMT); Benzotriazoles, for example, Drometrizole trisiloxane (DRT) and Methylene bis-benzotriazolyl tetramethylbutylphenol (MBP, biscotrizole); Dibenzoylmethane derivatives, for example, 4-tert-Butyl-4′-methoxydibenzoylmethane (BM-DBM, avobenzone); Cinnamates, for example, Ethylhexyl methoxycinnamate (OMC) and Isoamyl p-methoxycinnamate (IMC, amiloxate); and Camphor derivatives, for example, Terephtalydene dicamphor sulfonic acid (PDSA), 3-benzylidene camphor (3BC), Benzylidene camphor sulfonic acid (BCSA), 4-methylbenzylidene camphor (4-MBC), Polyacrylamidomethyl benzylidene camphor (PBC), and Camphor benzalkonium methosulfate (CBM).

In one embodiment of the disclosure, the sunscreen active agent comprises a photoprotecting effective amount of particulates of at least one inorganic pigment or nanopigment, non-limiting examples of which include titanium dioxide, zinc oxide, iron oxide, zirconium oxide, cerium oxide, or mixture thereof.

In an especially typical embodiment, the at least one sunscreen active agent is chosen from avobenzene, homosalate, octisalate, octocrylene, and oxybenzone.

Generally, the sunscreen active agent is present in the sunscreen formulation in amounts well-known in the art to be effective to protect a user to be exposed or already exposed to sunlight from the damaging effects of exposure to sunlight. Typically, these amounts range from 1-35% by weight, typically 3-25% by weight based on a total weight of the sunscreen formulation.

In one embodiment, the sunscreen active agent is present in the sunscreen formulation together with a booster, typically a UV booster, such as, merely for example, ethylhexyl methoxycrylene, diethylhexyl 2,6-Naphthalate, phenylethyl benzoate, and butyloctyl salicylate.

Additionally, the solvent can include an oil such as mineral or vegetable oil in varying amounts as a co-solvent or as described herein as “emollients”.

Emollients can include any appropriate oil, solvent, ester, triglyceride, etc. that is appropriate for the end use application. For suncare products, typical emollients include Triheptanoin, Isopropyl Palmitate, Triheptanoin (and) C13-C16 Isoparaffin, Heptyl Undecylenate, Caprylic/Capric Triglyceride, Diisooctyl Succinate, C13-C16 Isoparaffin (and) Heptyl Undecylenate, C12-C15 alkyl benzoate, Caprylic/Capric Triglyceride, Dibutyl Adipate, Butylene Glycol Dicaprylate/Dicaprate, and other appropriate esters. When diluted with emollient, the addition is done with the end-product held with mixing at around 80° C.-100° C. The combination is then further cooled to 50-70° C. for storage.

In one embodiment, the polymer is diluted to a final concentration of from 10 wt % to 99 wt % polymer, where the diluent is a suitable emollient for skin and sunscreen applications, consisting of an ester or triglyceride.

In another embodiment, the polymer is diluted to a final concentration of from 30 wt % to 90 wt % polymer, where the diluent is a suitable emollient for skin and sunscreen applications, consisting of an ester or triglyceride from the list given above.

In yet another embodiment, the polymer is diluted to a final concentration of from 50 wt % to 80 wt % polymer, where the diluent is a suitable emollient for skin and sunscreen applications, consisting of an ester or triglyceride.

The sunscreen formulation may additionally comprise other film-forming polymers in addition to the polyester described herein. Such other film-forming polymers can be chosen from, merely for example, starch ester-based polyglucose polymers, such as are described in U.S. Pat. No. 11,135,148, the entire contents of which are hereby incorporated by reference in various non-limiting embodiments; polyesters, such as are described in US 2021/0259930, US 2021/0259945, and US 2021/0259946, the entire contents of which are hereby incorporated by reference in various non-limiting embodiments; and N-alkyl (meth)acrylamide copolymers, such as are described in US 20180098930, the entire contents of which are hereby incorporated by reference in various non-limiting embodiments. Additional examples of suitable other film-forming polymers include film-forming polymer comprising at least 5% by weight, based on a total weight of the film-forming polymer, of an acid-containing monomer. In a typical embodiment, the film-forming polymer comprises at least 5% by weight of a carboxylic acid-containing monomer. Non-limiting examples of these monomers are acrylic acid, crotonic acid, methacrylic acid, maleic acid, itaconic acid, and combinations and mixtures thereof. Additional film-forming polymers, either synthetic or natural can be used with the acid-containing polymers described above. Non-limiting examples of these additional film forming polymers are: from Nouryon, AMPHOMER® and AMPHOMER® LV-71 polymers (octylacrylamide/acrylates/butylaminoethyl methacrylate copolymer), AMPHOMER® HC polymer (acrylates/octylacrylamide copolymer), BALANCE® 0/55 and BALANCE CR® polymers (acrylates copolymer), BALANCE® 47 polymer (octylacrylamide/butylaminoethyl methacrylate copolymer), RESYN® 28-2930 polymer (VA/crotonates/vinyl neodecanoate copolymer), RESYN® 28-1310 polymer (VA/Crotonates copolymer), FLEXAN® polymers (sodium polystyrene sulfonate), DynamX polymer (polyurethane-14 (and) AMP-Acrylates copolymer), RESYN® XP polymer (acrylates/octylacrylamide copolymer), STRUCTURE® 2001 (acrylates/steareth-20 itaconate copolymer) and STRUCTURE® 3001 (acrylates/ceteth-20 itaconate copolymer); from ISP, OMNIREZ-2000 (PVM/MA half ethyl ester copolymer), GANEX® P-904 (butylated PVP), GANEX® V-216 (PVP/hexadecene copolymer) GANEX® V-220 (PVP/eicosene copolymer), GANEX® WP-660 (tricontanyl PVP), GANTREZ® A425 (butyl ester of PVM/MA copolymer), GANTREZ® AN-119 PVM/MA copolymer, GANTREZ® ES 225 (ethyl ester of PVM/MA copolymer), GANTREZ® ES425 (butyl ester of PVM/MA copolymer), GAFFIX® VC-713 (vinyl caprolactam/PVP/dimethylaminoethyl methacrylate copolymer), GAFQUAT® 755 (polyquaternium-11), GAFQUAT® HS-100 (poly-quaternium-28), AQUAFLEX XL-306 (Polyimide-1), AQUAFLEX® SF-40 (PVP/Vinylcaprolactam/DMAPA Acrylates Copolymer), AQUAFLEX® FX-64 (Isobutylene/Ethylmaleimide/Hydroxyethylmaleimide Copolymer), ALLIANZ LT-120 (Acrylates/C1-2 Succinates/Hydroxy acrylates Copolymer), STYLEZE® CC-10 (PVP/DMAPA Acrylates Copolymer), STYLEZE® 2000 (VP/Acrylates/Lauryl Methacrylate Copolymer), STYLEZE® W-20 (Poly quaternium-55), Copolymer Series (PVP/Dimethylamino ethylmethacrylate Copolymer), ADVANTAGE S and ADVANTAGE LCA (VinylcaprolactamNP/Dimethylaminoethyl Methacrylate Copolymer), ADVANTAGE PLUS (VA/Butyl Maleate/Isobornyl Acrylate Copolymer), Antaron ECo (Ethylcellulose); from BASF, ULTRAHOLD STRONG (acrylic acid/ethyl acrylate/t-butyl acrylamide), LUVIMER® 100P (t-butyl acrylate/ethyl acrylate/methacrylic acid), LUVIMER 36D (ethyl acrylate/t-butyl acrylate/methacrylic acid), LUVIQUAT® HM-552 (polyquaternium-16), LUVIQUAT® HOLD (polyquaternium-16), LUVISKOL® K30 (PVP), LUVISKOL K90 (PVP), LUVISKOL VA 64 (PVP/VA copolymer), LUVISKOL VA73W (PVPNA copolymer), LUVISKOL VA, LUVISET® PUR (Polyurethane-1), LUVISET® Clear (VP/MethacrylamideNinyl Imidazole Copolymer), LUVIFLEX® SOFT (Acrylates Copolymer), ULTRA HOLD 8 Acrylates/Acrylamide Copolymer), LUVISKOL® Plus (Polyvinylcaprolactam), LUVIFLEX® Silk (PEG/PPG-25/25 Dimethicone/Acrylates Copolymer); from Amerchol, AMERHOLD® DR-25 (acrylic acid/meth acrylic acid/acrylates/methacrylates); from Rohm & Haas, ACUDYNE® 258 (acrylic acid/methacrylic acid/acrylates/methacrylates/hydroxy ester acrylates; from Mitsubishi and distributed by Clariant, DIAFORMER® Z-301, DIA FORMER Z-SM®, and DIAFORMER Z-400 (methacryloyl ethyl betaine/acrylates copolymer), ACUDYNE 180 (Acrylates/Hydroxyesters Acrylates Copolymer), ACUDYNE SCP (Ethylenecarboxyamide/AMPSA/Methacrylates Copolymer), and the ACULYN® rheological modifiers; from ONDEO Nalco, FIXOMER® A-30 and FIXOMER N-28 (INCI names: methacrylic acid/sodium acrylamidomethyl propane sulfonate copolymer); from Noveon, FIXATE® G-100 (AMP-Acrylates/Allyl Meth acrylate Copolymer), FIXATE PLUS (Polyacrylates-X), CARBOPOL® Ultrez 10 (Carbomer), CARBOPOL Ultrez 20 (Acrylates/C10-30 Alkyl Acrylates Copolymer), AVALURE AC® series (Acrylates Copolymer), AVALURE UR® series (Polyurethane-2, Polyurethane-4, PPG-17/IPDI/DMPA Copolymer); polyethylene glycol; water-soluble acrylics; water-soluble polyesters; polyacryl amides; polyamines; polyquaternary amines; styrene maleic anhydride (SMA) resin; polyethylene amine; from Covestro, Baycusan® C 2000 (Polyurethane solution in ethanol (INCI-Polyurethane-64)), Baycusan® eco E 1000 (water-based polyurethane), Baycusan® C 1010 (water-based polyurethane), Baycusan® C 1008 (water-based polyurethane), Baycusan® C 1001 (water-based polyurethane); from Inolex, Inolex Lexfilm Sun (INCI-Polyester-7 (and) Neopentyl Glycol Diheptanoate), Inolex Lexfilm Sun Natural MB (INCI-Capryloyl Glycerin/Sebacic Acid Copolymer), Inolex WetFilm MB (INCI-Trimethylpentanediol/Adipic Acid/Glycerin Crosspolymer), Inolex Lexfilm Spray (INCI-Polyester-10 (and) Propylene Glycol Dibenzoate) and Inolex Lexorez 100 MB (INCI-Adipic Acid/Diglycol Crosspolymer); and other conventional polymers that are polar solvent soluble or that can be made soluble through neutralization with the appropriate base.

In another embodiment, the sunscreen formulation comprises at least one biodegradable polyester film-forming polymer in addition to the polyester described herein.

In another embodiment, the sunscreen formulation comprises at least one biodegradable diisostearoyl polyglyceryl-3 dimer dilinoleate film-forming polymer in addition to the polyester described herein.

In an especially typical embodiment, the diisostearoyl polyglyceryl-3 dimer dilinoleate film-forming polymer comprises diisostearoyl polyglyceryl-3 dimer dilinoleate and caprylic/capric triglyceride.

When the sunscreen formulation comprises another film-forming polymer in addition to the polyester described herein, such other film-forming polymer may also be present in the sunscreen formulation in an amount of 0.1 to 10 wt % based on a total weight of the formulation.

In a typical embodiment, such other film-forming polymer is incorporated into sunscreen formulations in an amount of 0.5 to 5 wt % based on a total weight of the formulation.

In another typical embodiment, the combined amounts of all other such film-forming polymers and the polyester described herein total 0.1 to 10 wt % based on a total weight of the formulation.

In an especially typical embodiment, the combined amounts of all other such film-forming polymers and the polyester described herein total 0.5 to 5 wt % based on a total weight of the formulation.

The disclosed sunscreen formulations may include a wide range of additional, optional components which are referred to herein as “cosmetic components”, but which can also include components generally known as pharmaceutically active agents. The CTFA Cosmetic Ingredient Handbook, Seventh Edition, 1997 and the Eighth Edition, 2000, which is incorporated by reference herein in its entirety, describes a wide variety of cosmetic and pharmaceutical ingredients commonly used in skin care compositions, which are suitable for use in the compositions of the present disclosure. Examples of these functional classes disclosed in this reference include: absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), SPF boosters, waterproofing agents, and viscosity increasing agents (aqueous and nonaqueous).

The compositions of this disclosure can be applied to the skin as a liquid rub on, but are most commonly applied as a spray. However, the compositions are not limited to those compositions applied to the skin primarily as a sunscreen agent. The compositions also incorporate those formulations where the sunscreen active agent is an ingredient in another topically applied composition. Some non-limiting examples are lipstick, make-up, lip-balm, eye-shadow, hair dyes and conditioners, or any application where sun protection may be deemed beneficial.

In an embodiment, wherein the disclosed formulation is to be used in an application where sun protection may be deemed beneficial, the disclosed formulation affords a static sun protection factor (“static SPF”). Without being limiting, the SPF could be greater or lesser than 10, or greater than 20, or greater than 25, or greater than 30, or greater than 35, or greater than 40, or greater than 45, or greater than 50, or greater than 55, or greater than 60, or greater than 65, or greater than 70, or greater than 75, or greater than 80, or greater than 85, or even higher.

In another embodiment, the disclosed formulation affords a static SPF greater than 60.

In an embodiment, wherein the disclosed formulation is to be used in an application where sun protection may be deemed beneficial, the disclosed formulation affords a water resistance sun protection factor (“WR SPF”). Again, without being limiting, the SPF could be greater or lesser than 10, or greater than 20, or greater than 25, or greater than 30, or greater than 35, or greater than 40, or greater than 45, or greater than greater than 50, or greater than 55, or greater than 60, or greater than 65, or greater than 70, or greater than 75, or even higher.

In another embodiment, the disclosed formulation affords a WR SPF greater than 60.

The disclosure will now be described in greater detail with reference to the following non-limiting examples.

### EXAMPLES

### Example 1

To a 100 m 3-neck flask immersed in an oil bath with downward distillation set-up, magnetic stirrer, and thermometer with N2 inlet/atmosphere, were added the following raw materials: 28.8 grams of PolyGlycerol-3 (Inovyn/CAS #25618-55-7); 15.64 grams of dodecanedioic acid (Alfa Aesar/CAS #693-23-2); 6.77 grams of Radiacid 0960 (hydrogenated dimer acid from Oleon/CAS #68783-41-5); 24.56 grams of Radiacid 0161 (rape fatty acid from Oleon/CAS #68002-88-0); and 0.48 grams of NaOH (50% active). The temperature of the oil bath was adjusted to 180-200° C. After solid components of the reaction melted, the stirring rate was set to the highest possible setting to reduce potential foaming and the N2 inlet was set to start N2 bubbling. Oil bath temperature and magnetic stirrer rpms were gradually increased and acid value monitored until acid value was stable and as low as possible. When the acid value stabilized around 1.44 mg KOH/g, the reaction was stopped and the reaction mixture poured into a silicone tray. 71.69 grams of product polyester were obtained, as a non-tacky opaque-cloudy solid.

In a similar manner, suitable polyesters can be prepared from the following raw materials reacted in the indicated molar ratios:

TABLE 1

| | Polymer | | | | | | | | | |
|---|---|---|---|---|---|---|---|---|---|---|
| Ingredient | 2 | 3 | 4 | 5 | 6 | 7 | 8 | 9 | 10 | 11 |
| Polyglycerol | 3 | 3 | 3 | 3 | 3 | 3 | 3 | 3 | 3 | 3 |
| Dodecanedioic Acid | 1.5 | | 1.5 | | 1.5 | | 1.8 | 1.8 | | |
| Sebacic Acid | | 1.5 | | 1.5 | | 1.7 | | | 1.5 | 2 |
| Hydrogenated Dimer | 0.5 | 0.5 | 0.5 | 0.5 | 0.5 | 0.3 | 0.2 | 0.2 | | |
| Acid | | | | | | | | | | |
| N-Stearoyl Glutamic | | | | | | | | | 0.5 | |
| Acid | | | | | | | | | | |
| Rape Fatty Acid | | 2 | 2 | | | 2 | | | | 2 |
| Stearic Acid | | | | | | | | | 2 | |
| Behenic Acid 90% | 1 | | | | 2 | | 1 | 1 | | |
| Behenic Acid 47% | | | | 2 | | | | | | |
| Isostearic Acid | | | | | | | | 1 | | |
| Lauric Acid | 1 | | | | | | 1 | | | |

### Example 2

Anhydrous formulations were prepared to exemplify a spray product: Except as set forth in Table 2 below, an anhydrous concentrate was made in a 400 mL beaker, in which 63 grams anhydrous ethanol SDA-40B is charged to the beaker and with mixing, 2 g polyester derivative or comparative polymer is added and mixed for ˜30 minutes until completely dispersed. With continued mixing, an oil phase consisting of 3 g avobenzone (Neo Heliopan® 357, Symrise), 13 g homosalate (Neo Heliopan® HMS, Symrise), 5 g ethylhexyl salicylate (Neo Heliopan® OS, Symrise), 9 g octocrylene (Neo Heliopan® 303, Symrise), and 5 g C12-15 alkyl benzoate (Finsolv TN, Innospec) is added to the beaker and mixed for ˜30 minutes and the results observed.

TABLE 2

| | Gram | Weight | | |
|---|---|---|---|---|
| Ingredient | used | Percent | INCI or Chemical Name | CAS Number |
| Ethanol Phase | | | | |
| Anhydrous Ethanol | 63.00 | 63.00% | SD Alcohol 40B | 64-17-5 |
| Film-Forming | 2.00 | 2.00% | Polymer of this | |
| Polymer | | | Disclosure or | |
| | | | Comparative | |
| Oil Phase | | | | |
| NEO HELIOPAN ® | 3.00 | 3.00% | Avobenzone | 70356-09-1 |
| 357 | | | | |
| NEO HELIOPAN ® | 13.00 | 13.00% | Homosalate | 118-56-9 |
| HMS | | | | |
| NEO HELIOPAN ® | 5.00 | 5.00% | Octisalate | 118-60-5 |
| OS | | | | |
| NEO HELIOPAN ® | 9.00 | 9.00% | Octocrylene | 6197-30-4 |
| 303 | | | | |
| Finsolv ® TN | 5.00 | 5.00% | C12-15 Alkyl Benzoate | 68411-27-8 |
| Total Weight | 100 | 100.00% | | |

### Solubility Results

The desired result is a clear, one-phase, and complete solution, which is taken as an indication that the formulation is fully soluble. On the other hand, if the solution is hazy or includes precipitate, this is taken as an indication that the formulation is at least partly insoluble. Evaluations are made immediately after mixing (“Initial Solubility”) and 24 hours later (“24 Hour Solubility”).

TABLE 3

| | Film-Forming | Initial Formulation | 24 Hour Formulation |
|---|---|---|---|
| # | Polymer | Solubility | Solubility |
| 1 | Dermacryl ® 79¹ | Clear | Clear |
| 2 | SolAmaze ™² Natural | Clear | Clear |
| 3 | Polymer 1 | Clear | Clear |
| 4 | Polymer 2 | Clear | Clear |
| 5 | Polymer 3 | Clear | Clear |
| 6 | Polymer 4 | Clear | Clear |
| 7 | Polymer 5 | Clear | Clear |
| 8 | Polymer 6 | Clear | Clear |
| 9 | Polymer 7 | Clear | Clear |
| 10 | Polymer 8 | Clear | Clear |
| 11 | Polymer 9 | Clear | Clear |
| 12 | Polymer 10 | Clear | Clear |
| 13 | Polymer 11 | Clear | Clear |

¹Acrylic copolymer available from Nouryon
²Diisostearoyl Polyglyceryl-3 Dimer Dilinoleate (and) Caprylic/Capric Triglyceride available from Nouryon

The results demonstrate polymers prepared according to the present disclosure in a number of different manners can be formulated to form stable solutions in anhydrous ethanol.

Generally speaking, the solubility of any formulation including any disclosed film-forming polymer using any particular solvent or solvent system can be determined analogously to this example in a simple and straightforward manner.

### Example 3

Selected polymer samples (using the numbering in the tables above) were prepared in ethanol solvent with the oil phase and blended with propellant according to the following procedure to understand compatibility with propellants.

### Formulation Preparation Procedure:

1. Charge all ethanol in the formulation to the main mixing vessel.
2. Begin mixing with propeller agitation (adjust the speed of the speed of the mixing until there is a vortex pulled ⅔ of the way down the mixing shaft).
3. Slowly add the polymer powder by sifting it into the side of the vortex. Allow the polymer to disperse completely.
4. Slowly add the oil phase into the vortex and continue mixing until clear and homogeneous.
5. Fill concentrate into aerosol containers and charge with propellant.
6. Charged aerosols are observed for clarity, phases, and any precipitation that may have formed initially and over time.

In the following examples the propellant is isobutane at 30% of the total formulation. In each sample formulation reported herein, unless stated otherwise the formulations tested include 1.4 wt % polymer, ethanol QS to 44.1 wt %, 24.5 wt % UV oil phase, and 30 wt % propellant.

The desired result is a clear, one-phase, and complete solution, which is taken as an indication that the formulation is compatible. On the other hand, if the solution is hazy or opaque or includes precipitate, this is taken as an indication that the formulation is at least partly incompatible.

TABLE 4

| # | Film-Forming Polymer | 30% Isobutane |
|---|---|---|
| 1 | Dermacryl ® 79 | Clear |
| 2 | SolAmaze ™ Natural | Clear |
| 3 | Polymer 1 | Clear |
| 4 | Polymer 2 | Clear |
| 5 | Polymer 3 | Clear |
| 6 | Polymer 4 | Clear |
| 7 | Polymer 5 | Clear |
| 8 | Polymer 6 | Clear |
| 9 | Polymer 7 | Clear |
| 10 | Polymer 8 | Clear |
| 11 | Polymer 9 | Clear |
| 12 | Polymer 10 | Clear |
| 13 | Polymer 11 | Clear |

The results show that the disclosed film-forming polymers are compatible with isobutane propellant.

Generally speaking, the compatibility of any disclosed film-forming polymer and a particular propellant or solvent-propellant system can be determined analogously to this example in a simple and straightforward manner.

### Example 4

In order to demonstrate the effectiveness of the sunscreen formulations examples made with polyesters of this disclosure, or lack of effectiveness of formulations made with comparative products, for each formulation made, a series of tests are carried out to quantify the so-called Sun Protection Factor (SPF) and to demonstrate their effectiveness in creating a demonstrable water-resistant suncare formulation.

Due to the importance of SPF testing for labeling consumer suncare products, governmental agencies regulate the labeling of these products and require testing of the products on human panelists with specific statistical criteria. (Static) SPF measures the ratio of the time it takes for sunscreen protected skin to show the onset of erythema as compared to a non-sunscreen protected area of skin on the same human panelist.

Water-resistant (WR) SPF measurements likewise do that after a standard immersion of the skin in water at 40° C.

For laboratory measurements of SPF and WR SPF, a series of In-Vitro measurements can be made. Colipa Test methods are used in Europe. FDA 21 CFR Section 201 and 320 tests are used in the US. These measurements involved using plastic films or substrates to simulate the roughness and other properties of human skin. In this case, the substrate used SPF measurements can be taken using a variety of instruments. A Labsphere UV-1000 Spectrophotometer was used. Artificial skins including partially-hydrophilic acrylic polymer material, Vitro Skin™ from Florida Suncare, Inc., (formerly IMS Inc) were used following manufacturer instructions.

The film was coated with 20×5 mg drops of sunscreen concentrate, which were carefully and evenly distributed over the 55.8 cm2 area of the film, again coating with 2 mg/cm2. The UV spectrophotometer was used to measure the absorption (or transmission) of both UVA radiation (320-400 nm) and UVB radiation (290-320 nm). From these values the In-Vitro SPF could be measured for the skin with and without suncare product applied to the artificial skin.

The results are reported as In-Vitro SPF and In-Vitro WR SPF, where the former is again the measurement after a standard drying time and the latter is the measurement of the same film after 80 minutes immersion in water held at 40° C. These In-Vitro measurements have been shown to correlate to In-Vivo data. Other data such as UVA and UVB ratio can also be determined and used to quantify protection provided by the sunscreen formulations. Alternatively, for the Colipa Tech method, Helioplate HD6 embossed PMMA plates are available from Helio Labs Inc. These PPMA plates have a 6 micrometer root mean square surface roughness and are used without hydration. Due to the depth of the roughness, the same 2 mg/cm2 dose of sunscreen emulsion, once coated and dried on the plate, will have “peaks” of the surface roughness which are covered with relatively less UV absorbing oil. Thus, the absorbance and calculated SPF can be a bit reduced relative to in-vivo and other in-vitro measurement techniques. However, both in-vitro substrates give reproducible results and can be compared using appropriate correction factors.

Thus, as is standard in the art, UV absorption through plastic films coated with UV-absorbing suncare formulation is used to mimic human skin and is measured with a spectrophotometer to provide a measure of sun protection factor (SPF). SPF is simply the ratio of the initial light to the transmitted light through the UV absorbing film. If 100% is reduced to 10%, the SPF is 10. If 100% is reduced to 1%, the SPF is 100. SPF 50 corresponds to 2% of the UV light being transmitted through the skin.

In each case, a UV-absorbing film having a surface area of 55.8 cm2 was coated with 20×5 mg drops of a solution including the test polymer and subjected to UV light in the 280 to 400 nm range to provide a so-called “static” SPF measurement.

In-Vitro Water-Resistance SPF (WR SPF) measurements are taken from the same film after it is placed in a water-bath, heated to 40° C., and subjected to mild agitation. The sample after being withdrawn is gently dried under reproducible conditions and again measured for light transmission, this time providing WR SPF data.

TABLE 5

| # | Film-Forming Polymer | Static SPF | WR SPF |
|---|---|---|---|
| 1 | Dermacryl ® 79 | 56.6 | 55.8 |
| 2 | SolAmaze ™ | 33.2 | 20.3 |
| 3 | Polymer 1 | 57.9 | 64.7 |
| 4 | Polymer 2 | 42.8 | 49.9 |
| 5 | Polymer 3 | 58.5 | 54.7 |
| 6 | Polymer 4 | 62.3 | 50.6 |
| 7 | Polymer 5 | 58.4 | 52.3 |
| 8 | Polymer 6 | 50.7 | 62.0 |
| 9 | Polymer 7 | 59.9 | 57.6 |
| 10 | Polymer 8 | 48.1 | 56.7 |
| 11 | Polymer 9 | 51.3 | 51.6 |
| 12 | Polymer 10 | 57.9 | 64.7 |
| 13 | Polymer 11 | 63.5 | 35.1 |

### Example 5

Pair Comparison—blind evaluations are performed by 8 panelists using two different leave-on products by applying them to their volar forearm. The panelist has to select one product as applied to their volar forearm as more intense than the other in each performance attribute. Performance data is summarized and analyzed statistically.

### Skin Preparation:

Panelists will clean the entire volar forearm area as well as their evaluation fingers using provided ethanol wipes. Once the ethanol has fully evaporated an approximately 1.50″ diameter circle will be drawn on the forearm as the evaluation site and labelled alphabetically (A or B). Use caution to avoid the wrist and crook of the arm areas and provide sufficient space between samples as to ensure they don't run into one another during application.

### Application Evaluation by Panelists:

1. Provide the panelists with the evaluation ballot for recording their answers
2. Making sure the panelists are unaware of the identification of the sample, an electronic pipette should be used to apply approximately 0.4 μL of each sample (A and B) to the center of the evaluation sites.
3. The panelists spread the measured amount of product within the circle using the index or middle finger, using a gentle circular motion—stroking at a rate of approximately 2 strokes per second.
4. Indicated below are the attributes that panelists evaluated for comparison of the experimental sunscreen solution vs. a control sunscreen solution (DERMACRYL® 79; Acrylates/Octylacrylamide Copolymer, Nouryon) in the sunscreen formulation as outlined above:

### Spreadability: Ease of Moving Product Over the Skin (Difficult/Drag to Easy/Slip)

After 10 Rubs, Evaluate and Choose which Product has the Highest Intensity of the Following:
Stickiness: Degree to which fingers adhere to residual product (not sticky to very sticky)

### Visually Analyze the Forearm Test Site for Evaluate for:

Gloss: Amount or degree of light reflected off skin (none to high amount)
   Immediate after-Feel: Stroke Cleansed Fingers (1-2 Strokes) Lightly Across Skin and Evaluate for:
Slipperiness: Ease of moving fingers over the skin (difficult/drag to easy/slip)
Amount of Residue: Amount of product on skin (none to large amount)
Oiliness: A slippery, smooth, continuous feel (i.e. baby oil)
Powderiness: A thin, slippery coating that is very dry (i.e. corn starch)
   Assess Stickiness Again, after Product has had Time to Absorb into the Skin After ˜5 Minutes:
Stickiness2: Degree to which fingers adhere to residual product (not sticky to very sticky)

At the end of the attributes assessment, panelists are asked to select which product was typical, indicated by “overall preference”.

### Analysis of Data/Assessments:

Inferior Sample: experimental typical 0 to 1 times out of 8
No statistical difference: experimental sample typical 2 to 6 times out of 8
Superior Sample: experimental typical 7 to 8 times out of 8

TABLE 6

| | Film-Forming Polymer | | | | | | | |
|---|---|---|---|---|---|---|---|---|
| | Sensory Properties Compared to Dermacryl ® 79 | | | | | | | |
| Property | #2 | #3 | #4 | #5 | #6 | #8 | #9 | #10 |
| Spreadability | 7 | 2 | 4 | 4 | 3 | 4 | 5 | 4 |
| Stickiness | 3 | 3 | 3 | 2 | 2 | 6 | 5 | 3 |
| Gloss | 5 | 6 | 6 | 3 | 4 | 2 | 5 | 4 |
| Slipperiness | 3 | 5 | 5 | 3 | 4 | 4 | 3 | 5 |
| Residue | 6 | 7 | 4 | 3 | 4 | 7 | 6 | 7 |
| Oily | 7 | 7 | 5 | 8 | 6 | 8 | 7 | 7 |
| Powdery | 1 | 1 | 2 | 1 | 2 | 0 | 1 | 1 |
| Stickiness 2 | 6 | 6 | 6 | 6 | 4 | 8 | 6 | 6 |
| Preference | 1 | 0 | 2 | 3 | 3 | 0 | 2 | 3 |

### Example 6

Several examples of this disclosure were tested by OECD biodegradability method 301D, details of which are readily available. Testing was performed in both river water and activated sludge. [OECD (1992), Test No. 301: Ready Biodegradability, OECD Guidelines for the Testing of Chemicals, Section 3, OECD Publishing, Paris, https://doi.org/10.1787/9789264070349-en.]

TABLE 7

| | |
|---|---|
| | Biodegradability in River Water |
| Biodegradation | Film-Forming Polymer |

| percentage at Day X | #1 | #2 | #7 | #9 | #10 |
|---|---|---|---|---|---|
| 7 | 47 | 32 | 39 | 46 | 29 |
| 14 | 57 | 49 | 49 | 58 | 50 |
| 21 | 65 | 53 | 58 | 63 | 60 |
| 28 | 67 | 56 | 60 | 62 | 63 |
| 42 | — | 56 | — | — | 64 |
| 56 | — | 56 | — | — | 64 |
| 84 | — | — | — | — | — |

TABLE 8

| | Biodegradability in Activated Sludge | | | | |
|---|---|---|---|---|---|
| Biodegradation | Film-Forming Polymer | | | | |
| percentage at Day X | #1 | #2 | #7 | #9 | #10 |
| 7 | 39 | 24 | 32 | 40 | 34 |
| 14 | 47 | 43 | 43 | 48 | 60 |
| 21 | 53 | 47 | 47 | 50 | 69 |
| 28 | 54 | 52 | 48 | 51 | 75 |
| 42 | — | 55 | — | — | 79 |
| 56 | — | 54 | — | — | 77 |
| 84 | — | — | — | — | — |

The 301D ratings include “Readily Biodegradable” meaning 60% to 100% biodegradation in 28 days or less. “Inherently Biodegradable” means 20% to 60% biodegradation in 28 days, and “Non-Biodegradable” means less than 20% biodegradation in 28 days.

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A sunscreen formulation comprising:
(a) a volatile solvent; and
(b) at least one film-forming polymer that is the reaction product of:
(i) at least one polyglycerol;
(ii) at least one diacid having 4-30 carbon atoms; and
(iii) at least one fatty acid having 8-30 carbon atoms.

2. The sunscreen formulation according to claim 1, wherein the volatile solvent comprises at least one C₁₋₆ straight or branched chain alcohol.

3. The sunscreen formulation according to claim 1, wherein the volatile solvent comprises ethanol.

4. The sunscreen formulation according to claim 1, which includes from about 1 to about 3% by weight of water.

5. The sunscreen formulation according to claim 1, which does not include water.

6. The sunscreen formulation according to claim 5, which comprises ethanol but not does include any other alcohol.

7. The sunscreen formulation according to claim 1, which further comprises at least one sunscreen active agent chosen from para aminobenzoic acid, avobenzone, cinoxate, dioxybenzone, homosalate, menthyl anthranilate, octyl salicylate, oxybenzone, padimate O, phenylbenzimidazole sulfonic acid, sulisobenzone, trolamine salicylate, titanium dioxide, zinc oxide, diethanolamine methoxycinnamate, digalloy trioleate, ethyl dihydroxypropyl PABA, glyceryl aminobenzoate, lawsone with dihydroxyacetone, red petrolatum, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, bis-ethylhexyloxyphenol methoxyphenyltriazine, 4-methylbenzylidenecamphor, homosalate, butyl methoxydibenzoylmethane, octocrylene, octyl salicyclate, bemotrizinol and isopentyl 4-methoxycinnamate, benzophenone-3 (BP3), benzophenone-4 (BP4), homosalate (HMS), 2-ethylhexyl salicylate (EHS), ethylhexyl dimethyl PABA (OD-PABA), 4-p-aminobenzoic acid (PABA), phenylbenzimidazole sulfonic acid (PMDSA), disodium phenyl dibenzimidazole tetrasulfonate (bisdisulizole disodium), ethylhexyltriazone (OT), diethylhexyl butamido triazone (DBT), bis-ethylhexyloxyphenol methoxyphenyl triazine (EMT), drometrizole trisiloxane (DRT), methylene bis-benzotriazolyl tetramethylbutylphenol (MBP, biscotrizole), 4-tert-butyl-4′-methoxydibenzoylmethane (BM-DBM, avobenzone), ethylhexyl methoxycinnamate (OMC), isoamyl p-methoxycinnamate (IMC, amiloxate), terephtalydene dicamphor sulfonic acid (PDSA), 3-benzylidene camphor (3BC), benzylidene camphor sulfonic acid (BCSA), 4-methylbenzylidene camphor (4-MBC), polyacrylamidomethyl benzylidene camphor (PBC), camphor benzalkonium methosulfate (CBM), titanium dioxide, zinc oxide, iron oxide, zirconium oxide, cerium oxide, and mixtures thereof.

8. The sunscreen formulation according to claim 7, wherein the at least one sunscreen active agent is chosen from avobenzene, homosalate, octisalate, octocrylene, oxybenzone, and combinations thereof.

9. The sunscreen formulation according to claim 1, which further comprises an emollient.

10. The sunscreen formulation according to claim 9, wherein the emollient is chosen from alkyl benzoates, capric triglycerides, and combinations thereof.

11. The sunscreen formulation according to claim 1, wherein the at least one diacid is chosen from dodecanedioic acid, sebacic acid, N-stearoyl glutamic acid, and mixtures thereof; and the formulation optionally additionally comprises a C36 or higher dimer acid.

12. The sunscreen formulation according to claim 11, which additionally comprises a C36 or higher dimer acid.

13. The sunscreen formulation according to claim 11, which does not comprise a C36 or higher dimer acid.

14. The sunscreen formulation according to claim 1, which is a sprayable sunscreen formulation.

15. The sunscreen formulation according to claim 14, which further comprises one or more additives chosen from plasticizers, UV absorbers, dyes, perfumes, preservatives, viscosity modifiers, vitamins, moisturizers, anti-itch ingredients, and combinations thereof.

16. The sunscreen formulation according to claim 1, which is an aerosol formulation, and further comprises a propellant.

17. The sunscreen formulation according to claim 16, wherein the propellant is a hydrocarbon.

18. The sunscreen formulation according to claim 17, wherein the hydrocarbon is isobutane.

19. A sunscreen formulation comprising:
(a) an alcohol-based solvent system comprising ethanol;
(b) at least one film-forming polymer that is the reaction product of:
(i) at least one polyglycerol;
(ii) at least one diacid having 4-30 carbon atoms; and
(iii) at least one fatty acid having 8-30 carbon atoms;
(c) at least one sunscreen active agent;
(d) about 0 to about 3% by weight of water;
(e) optionally at least one emollient;
(f) optionally at least one C36 or higher dimer acid;
(g) optionally at least one propellant; and
(h) optionally one or more additives chosen from plasticizers, UV absorbers, dyes, perfumes, preservatives, viscosity modifiers, vitamins, moisturizers, anti-itch ingredients, and combinations thereof.

20. A method comprising the step of applying an amount the sunscreen formulation according to claim 1 to skin.
